(19) United States
(12) Patent Application Publication
(10) Pub. No.: US 2002/0044951 A1
(43) Pub. Date: Apr. 18, 2002

(54) Isolated and purified nonpeptide antigens from mycobacterium tuberculosis

(76) Inventors: Gui Liu, Medford, MA (US); Gerald Beltz, Lexington, MA (US); Kenneth LeClair, Needham, MA (US); Daniel Cox, Medway, MA (US); Charlotte Kensil, Milford, MA (US)

Correspondence Address: PENNIE AND EDMONDS, 1155 AVENUE OF THE AMERICAS, NEW YORK, NY, 100362711

(21) Appl. No.: 09/825,789

(22) Filed: Apr. 4, 2001

Related U.S. Application Data
(60) Provisional application No. 60/194,519, filed on Apr. 4, 2000.

(51) Int. Cl. A61K 39/02; A61K 31/715

(52) U.S. Cl. 424/264.1; 514/54

(57) ABSTRACT

Nonpeptide antigens were isolated and purified from Mycobacterium tuberculosis. The antigens were used in vaccine compositions, pharmaceutical compositions and methods to elicit an immune response to Mycobacterium tuberculosis in a mammal.

### GOVERNMENT SUPPORT

This invention was supported, in whole or in part, by grant R43/44-AI-40798 from the National Institutes of Health. The Government has certain rights in the invention. This application claims benefit of U.S. Provisional Application No. 60/194,519, filed Apr. 4, 2000, incorporated herein by reference.

### FIELD OF THE INVENTION

The invention described herein is related to vaccine compositions that are used to elicit an immune response specific for Mycobacterium tuberculosis in a mammal, as well as methods to elicit the immune response specific for Mycobacterium tuberculosis. The invention also pertains to immunogenic or vaccine compositions comprising at least one nonpeptide antigen isolated from Mycobacterium tuberculosis, wherein the isolated nonpeptide antigen elicits a specific immune response against Mycobacterium tuberculosis, and may further comprise one or more T-cell stimulating compounds. Several inventive isolated and purified nonpeptide antigens from the mycobacteria Mycobacterium tuberculosis are described herein.

### BACKGROUND OF THE INVENTION

Immunity from a bacterial pathogen may be mediated by a protein, but may also be mediated by a nonpeptide antigen such as a polysaccharide or a lipid. Some lipid, glycolipid, and phospholipid antigens have already been identified from various Mycobacterium species. These include mycolic acid, inositol-containing phospholipids (e.g., lipoarabinomannan (LAM) and phosphatidylinositol mannosides (PIMs)), and mycolyl glycolipids (e.g., glucose monomycolate (GMM)), all of which may be purified from mycobacterial cell walls (Beckman, et al., Nature 372:691 (1994); Sieling, et al., Science 269:227 (1995); and Moody, et al., Science 278:283 (1997)). Some of these molecules share similar structural features consisting of a relatively hydrophilic polar head group linked to a single or dual branched hydrophobic acyl chain(s). It has been hypothesized that these molecules may be presented by the CD1 pathway of antigen presentation. A nonpeptide antigen (e.g., from bacteria or parasites) is ingested by an antigen presenting cell (“APC”) (e.g., macrophage, B-cell or dendritic cell) and may then be presented in conjunction with a CD1 molecule, to thereby induce T-cell proliferation. (Porcelli, et al., Current Opinion in Immunology 8:510-516 (1996)). This pathway is referred to herein as the “CD1 antigen-presenting pathway.” These lipid or nonpeptide antigens are processed independently from the major histocompatability complex (“MHC”) peptide antigen-presenting pathway. The immune response that results from the CD1 antigen presenting pathway is referred to as a “CD1-restricted response.” The immunologic role of CD1 presentation of hydrophobic nonpeptide antigens has been demonstrated in M. tuberculosis infection. The presentation of mycobacterial lipid and glycolipid antigens by CD1 molecules initiates an MHC-independent pathway of host defense against mycobacterial infection in vivo by both cytolytic- and cytokine-based mechanisms. Specifically, CD8⁺ or CD8⁻/CD4⁻ (double negative) T-cells recognize nonpeptide microbial antigens when presented in the context of CD1 molecules. These CD1-restricted T-cells are cytolytic and kill mycobacterial infected monocytes (Stenger, et al., Science 276:1684 (1997)). CD8⁺, CD1-restricted M. tuberculosis specific T-cell lines derived from the blood of human donors produce Th1-type cytokines, such as interferon, which may facilitate control of mycobacterial infections, and may play a role in clearing intracellular microbial infections.

Nonpeptide antigens may also induce an immune response through an MHC-dependent pathway of presentation, for example through Class II MHC, particularly if it is presented in association with a protein antigen. It is well-known that a potent immune response can be raised to a nonpeptide antigen that is conjugated to a protein carrier such as keyhole limpet hemocyanin that provides T-cell help and cytokine stimulation through T-cell recognition of carrier peptide bound to Class II MHC on APC. An example is the T-cell dependent immune response raised against T-independent pneumococcal polysaccharide antigens chemically conjugated to a T-cell dependent protein carrier such as tetanus toxoid, CRM₁₉₇ (nontoxic mutant diptheria toxin), or OMPC (outer membrane protein complex of Neisseria meningitidis) (R. Eby, Pharmaceutical Bioechnology 6: 695-714(1995). Another example is the T-cell dependent immune response to LOS (lipooligosaccharide) from nontypeable Haemophilus influenzae after conjugation to a protein carrier (Gu, et al., Vaccine 18:1264-1272 (2000)).

The results disclosed herein demonstrate compounds from M. tuberculosis that may be utilized as vaccine candidates. These compounds may be tested as vaccine candidates in a variety of ways. They may be tested for their potential to induce T-cell antigen-specific cytolytic or proliferative responses. They may also be tested for their potential to induce compound-specific antibodies. Moreover, the vaccine compositions described herein may be tested for reduction of colony counts in animals vaccinated with the novel M. tuberculosis compounds compared to negative control groups after a challenge with M. tuberculosis. The vaccines of the present invention may be used both prophylactictically as well as therapeutically.

### SUMMARY OF THE INVENTION

The invention pertains to inventive compositions consisting of nonpeptide antigens isolated and purified from M. tuberculosis. Also, the present inventive compositions may also comprise the nonpeptide antigen with a T-cell stimulating compound, such as an adjuvant with which it is mixed or a protein carrier to which it is chemically conjugated, to elicit an immune response to the antigen. Hence, the invention relates to vaccine compositions comprising one or more antigens isolated from M. tuberculosis in which a T-cell stimulating compound or compounds may be included. These nonpeptide antigens may be developed into vaccine candidates. Similarly, the invention encompasses methods of using these compositions to elicit an immune response to M. tuberculosis such that the vaccine may be used as a prophylactic or therapeutic treatment. The invention also relates to vaccine compositions comprising a lipid antigen and a lipid carrier, wherein the composition is sonicated or vortexed, followed by extrusion through a membrane of, for example, 400nm or 800 nm pore size. Adjuvants are optionally added to the extruded vaccine compositions. The invention also relates to method of making such vaccine compositions.

### DESCRIPTION OF THE FIGURES

FIG. 1 shows the analytic HPLC profile of the isolated and purified compound 2640-35A (FIG. 1A) and a blank run (FIG. 1B).

FIG. 2 is the ¹H-NMR profile of the isolated and purified compound 2640-35A.

FIG. 3 provided the ¹³C-NMR profile of the isolated and purified compound 2640-35A.

FIG. 4 gives the analytic HPLC profile of the isolated and purified compound 2640-38C (FIG. 4A) and a blank run (FIG. 4B).

FIG. 5 gives the analytic ¹H-NMR profile of the isolated and purified compound 2640-47F.

FIG. 6 is the ¹³C-NMR profile of the isolated and purified compound 2640-47F.

FIG. 7 provides the ¹H-NMR profile of the isolated and purified compound 2640-48D.

FIG. 8 gives the ¹³C-NMR profile of the isolated and purified compound 2640-48D.

FIG. 9 provides the ¹H-NMR (400 MHz, CDCl₃) profile of the isolated and purified compound NA-2-2

FIG. 10 provides the negative ESI-MS spectrum of NA-2-12-1

FIG. 11 provides the negative ESI-MS/MS spectra of NA-2-12-1

FIG. 12 provides the the ¹H-NMR (400 MHz, CDCl₃) profile of NA-2-12-1

FIG. 13 provides the proliferation results of PBMC to lipid antigen subfractions

### DESCRIPTION OF THE PREFERRED EMBODIMENTS

In particular, mycobacteria are a genus of aerobic intracellular bacterial organisms which upon invasion of their host, survive within endosomal compartments of monocytes and macrophages. Human mycobacterial diseases include tuberculosis (caused by M. tuberculosis), leprosy (caused by M. leprae), Bairnsdale ulcers (caused by M. ulcerans) and various infections caused by M. Marimum, M. kansaii, M. scrofulaceum, M. szulgai, M. xenopi, M. fortitum, M. chelonei, M. haemophilum and M. intracellulare. Wolinsky, E., Chapter 37 in Microbiology: Including Immunology and Molecular Genetics, 3^{rd} Ed., Harper & Row, Philadelphia (1980); Daniel, T. M., Miller, R. A. and Freedman, S. D., Chapters 119, 120 and 121 respectively, in Harrison's Principles of Internal medicine, 11^{th} Ed., McGraw-Hill, New York (1987).

With the advent of AIDS, tuberculosis is increasing at a nearly logarithmic rate, and multidrug resistant strains are appearing and now account for one third of all cases in New York City. Bloom, et al., Science 257:1055 (1992); U.S. Congress, Office of Technology Assessment, The Continuing Challenge of Tuberculosis, OTA-H-574, U.S. Government Printing Office, Washington, D.C., (1993). Mycobacterial strains which were previously considered to be nonpathogenic strains (e.g., M. avium) have now become major killers of immunosuppressed AIDS patients. Moreover, current mycobacterial vaccines are either inadequate, or unavailable as in the case of M. leprae. Kaugmann S., Microbiol. Sci. 4:324-328 (1987); U.S. Congress, Office of Technology Assessment, The Continuing Challenge of Tuberculosis, pp. 62-67, OTA-H-574, U.S. Government Printing Office, Washington, D.C., (1993). The present invention's methods and compositions that boost the immune system are important in fighting such infectious conditions.

An “antigen” as used herein is a molecule or composition of matter that induces an immune response in an animal and interacts specifically with one or more antigen-recognizing components of an animal's immune system.

“Isolated” and “purified” as used herein to describe certain molecules, proteins, polysaccharides, lipids, antigens, and the like, refer to a state beyond that in which the molecules, proteins, polysaccharides, lipids, or antigens exist naturally in cells. In preferred embodiments, the isolated molecules, proteins, polysaccharides, lipids, antigens, and the like, are separated from greater than 20%, 30%, 40%, 50%, 60%, 70%, 80%, 90%, 95% or 99% of the proteins and/or the lipids with which the molecules, proteins, polysaccharides, lipids, antigens, and the like are normally associated naturally in cells. If the isolated molecules, proteins, polysaccharides, lipids, antigens, and the like are synthesized, they are contaminated with less than 50%, 40%, 30%, 20%, 10%, 5%, 1% or 0.1% of the chemical precursors or synthesis reagents used to synthesize the lipid antigen. In preferred embodiments, the molecules, proteins, polysaccaharides, lipids or antigens are at least 1% pure, 5% pure, 10% pure, 20% pure, 30% pure, 40% pure, 50% pure, 60% pure, 70% pure, 80% pure, 90% pure, 95% pure, 99% pure, or 100% pure. As used herein, the term “% pure” indicates the percentage of the total composition that is made up of the molecule of interest, by weight. Thus, a composition of 100 grams containing 50 grams of a molecule of interest is 50% pure with respect to the molecule of interest.

The term “nonpeptide antigen” refers to any antigen that does not contain a peptide bond, including, but not limited to, a polysaccharide antigen and a lipid antigen.

The term “lipid” as used herein refers to a wide variety of compounds that are characteristically poorly soluble in water. These may include fatty acids, neutral fats such mono, di-, and triglycerides, glyceryl ethers, glycosyl glycerols, phosphoglycerides such as phosphatidylcholine, phosphatidylethanolamine, and phosphatidylserine, phosphatidylglycerols, phosphatidylinositols, sphingolipids, sulfatides, diol lipids, terpenes, sterols, glycolipids, gangliosides, ceramides, cerebrosides, cerebroside sulfatides, and lipooligosaccharides. They may contain a polar headgroup such as a phosphate group, amino group, or saccharide.

The term “CD1 protein” and “CD1 target protein” and “CD1 molecule” as used herein refer to a collection of proteins that have been identified by their nucleotide and amino acid sequences, and by their structure, immunologic cross-reactivity, and/or distribution as being related to known CD1 molecules. A specific CD1 protein may be referred to as a member of the CD1 family of proteins. Members of the family include CD1a, CD1b, CD1c, CD1d, and CD1e.

The terms “CD1 ligand” and “CD1 antigen” and “CD1-presented antigen” herein refer to a ligand or an antigen that binds to a member of the CD1 family of proteins and causes the induction of an immune response. CD-1 antigens are the subject of U.S. Pat. Nos. 5,853,737, 5,679,347, each of which is incorporated herein by reference in its entirety for all purposes.

The term “vaccine composition” herein refers to a composition capable of producing an immune response. In a preferred embodiment, a vaccine composition, according to the invention, would produce immunity against disease in an animal. Preferably, the vaccine composition stimulates immunity and, more preferably, the vaccine composition enhances antibody production to an antigen and enhances induction of a cell-mediated immune response to the CD1 ligand. Alternatively, a vaccine composition may stimulate an antibody response depending on the particular disease or microbial infection.

The term “immunotherapy” as used herein means a treatment to an individual that influences an immune response by immunopotentiation (i.e., enhancement) or immunosuppression (i.e., suppression). Enhancement of an immune response to the CD1 protein/CD1 ligand complex may occur by an upregulation of antibody production, an increase in cytotoxic T-cell responses, and stimulation of certain lymphokines and/or cytokines. In contrast, inhibition of an immune response may occur by downregulation of antibody production, a decrease in cell mediated response, and stimulation of certain suppressive cytokines.

A “T-cell stimulating compound” as used herein is a compound that may increase T-cell proliferation or T-cell helper effects when introduced to the immune system with an antigen. T-cell stimulating compounds include adjuvants and protein carriers. An “adjuvant” is a substance which enhances the immunogenic response to an antigen. In particular, adjuvants are generally considered to be in one of two categories: (1) “depot” adjuvants that help transport and retain antigens in lymphoid tissues, or (2) “immuno-modulators” that stimulate locally secreted cytokines. Adjuvants may be composed of bacteria or bacterial products which contribute to the immunogenicity of the antigen. Examples of adjuvants are mineral salt adjuvants (i.e., alum or calcium-based adjuvants), Incomplete or Complete Freund's Adjuvant, Bacille Calmette-Guerin (BCG) adjuvant, block polymer adjuvant (Titermax), cholera toxin (Cholera toxin-B-subunits, enterotoxin (LT) mutants), cytokines, CpG motif-containing adjuvants, oil/water emulsion adjuvants (oil and water; water and oil; water, oil and water adjuvants), MF-59 adjuvants, LeIF adjuvants (i.e., protein-based), liposome adjuvant, ISCOM adjuvant, Monophosphoryl lipid A adjuvant (MPL), biodegradable microsphere adjuvant, muramyl dipeptide adjuvant, polyphosphazene adjuvant, and a saponin adjuvant (including, but not limited to QS-7, QS-17, QS-18, or QS-21, QuilA, or active synthetic or semisynthetic derivatives). Preferably, the compositions comprise one or more adjuvants. A protein carrier is a protein that contains peptide or peptides that stimulate Class II MHC or Class I MHC. This stimulation of Class II MHC or Class I MHC increases the immune response to antigens that are chemically conjugated to the protein carrier. Examples of protein carriers are keyhole limpet hemocyanin, diptheria toxoid, tetanus toxoid, etc.

In a first aspect of the invention, the invention is directed to nonpeptide antigens isolated and purified from the mycobacterium M. tuberculosis. These antigens may be isolated from a lipid extract of M. tuberculosis. Preferably the lipids may be crudely fractionated nonpeptide compounds associated with different polarity classes of lipids. These may be fractionated into different polarity classes by fractionation with solvents of different polarity on a silica gel column or similar means (for example, fractionation into nonpolar classes with hexane or chloroform, intermediate polarity with acetone, and higher relative polarity with methanol). These different polarity classes may be further fractionated by normal phase or reversed phase HPLC to yield purified nonpeptide compounds. Preferably, the purified nonpeptide antigens described herein are selected from nonpolar, intermediate polarity, and high polarity classes. More preferably, the purified nonpeptide antigens are 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, or NA-2-17. In another, M-2, NA-2, or the total lipid fraction from M.tuberculosis may also be used as antigens. Antigen 2640-35A is also referred to as A-5-7, antigen 2640-38C is also referred to as A-5-9, antigen 2640-47E is also referred to as C-4-5, antigen 2640-47F is also referred to as C-4-6, antigen 2640-47H is also referred to as C-4-8, and antigen 2640-48D is also referred to as C-4-11.

In a preferred embodiment, the purified nonpeptide antigen comprises or consists of the compound of formula I.

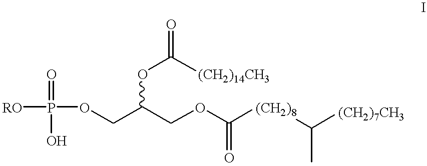

In a preferred embodiment, the purified nonpeptide antigen comprises or consists of the compound of formula II.

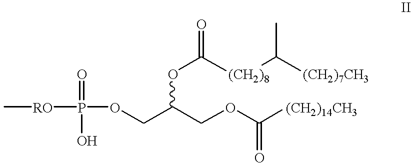

In a preferred embodiment, the purified nonpeptide antigen comprises or consists of the compound of formula III.

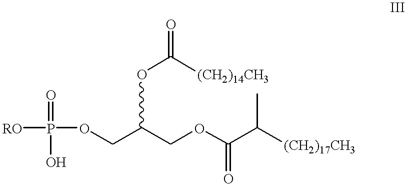

In a preferred embodiment, the purified nonpeptide antigen comprises or consists of the compound of formula IV.

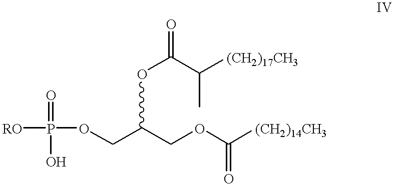

In a preferred embodiment, the purified nonpeptide antigen comprises or consists of the compound of formula V.

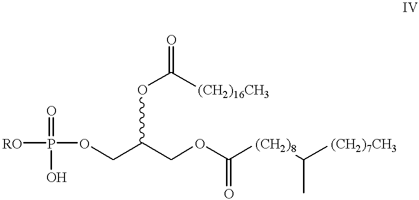

In a preferred embodiment, the purified nonpeptide antigen comprises or consists of the compound of formula VI.

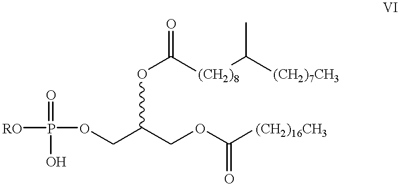

For each of the structures above, R is H, an amino acid, a peptide, or a carbohydrate. Preferably, R is H.

In a second aspect of the invention, the invention encompasses a vaccine composition comprising at least one nonpeptide antigen isolated and purified from M. tuberculosis. In another embodiment, the vaccine composition may further comprise at least one T-cell stimulating compound. Preferably, the nonpeptide antigen is selected from 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, or NA-2-17. In another embodiment, M-2, NA-2, or the total lipid fraction from M.tuberculosis may also be used as antigens. In another embodiment, the antigen comprises or consists of a compound of formula I. In another embodiment, the antigen comprises or consists of a compound of formula II. In another embodiment, the antigen comprises or consists of a compound of formula III. In another embodiment, the antigen comprises or consists of a compound of formula IV. In another embodiment, the antigen comprises or consists of a compound of formula V. In another embodiment, the antigen comprises or consists of a compound of formula VI. The lipid antigens of the invention may be obtained from any source, or may be synthesized.

The T-cell stimulating compound may preferably be either an adjuvant or a protein carrier. The adjuvant, more preferably, may be selected from mineral salt adjuvants, Incomplete or Complete Freund's Adjuvant, Bacille Calmette-Guerin adjuvant, block polymer adjuvant, cholera toxin, cytokine, CpG motif-containing adjuvants, oil/water emulsion adjuvants, MF-59 adjuvants, LeIF adjuvants, liposome adjuvant, ISCOM adjuvant, Monophosphoryl lipid A adjuvant, biodegradable microsphere adjuvant, muramyl dipeptide adjuvant, polyphosphazene adjuvants, and saponin adjuvants. The vaccine composition may further consist of conjugation of the nonpeptide antigen to a protein carrier that stimulates Class II MHC or Class I MHC. Examples of protein carriers are keyhole limpet hemocyanin, diptheria toxoid, tetanus toxoid, heat shock protein, etc. Most preferably, the vaccine composition comprises of a chemical conjugation of the lipid antigen to a protein carrier and mixing this conjugate with one or more adjuvants. The vaccine composition may be used to elicit or boost an immune response to M. tuberculosis in a mammal.

The inventive compositions disclosed herein may utilize synthetically derived nonpeptide antigens that utilize the CD1 -restricted pathway, the Class II MHC pathway, or the Class I MHC pathway if conjugated to a protein carrier A synthetic antigen, in accordance with this invention, is an antigen which is not naturally occurring in an organism. A synthetic antigen may be one that is chemically synthesized, or parts thereof are synthesized by combining elements, molecules or compounds. Further, a synthetic antigen may be constructed by combining two or more components, one or more of which is isolated or derived from an organism, thus producing a hybrid or chimeric antigen.

A synthetic antigen, may be, for example, an antigen comprising a lipid moiety and a hydrophilic moiety which is chemically modified or synthesized. Lipid antigens typically have a hydrophobic, lipid domain as well as a hydrophilic domain. The hydrophilic domain of a lipid antigen may be, but is not limited to, a carbohydrate (e.g., a sugar moiety), a carboxylic acid, a phosphate, or a carbohydrate and phosphate covalently bonded together. In a preferred embodiment, the synthetic antigen is comprised of a lipid domain from 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, or NA-2-17, and a hydrophilic domain not associated with said lipid portion in a naturally occurring lipid antigen found in wild-type Mycobacterium tuberculosis. Alternatively, in another embodiment, the lipid domain may be from M-2, NA-2, or the total lipid fraction from M. tuberculosis. In another embodiment, the lipid domain may be from a compound of formula I. In another embodiment, the lipid domain may be from a compound of formula II. In another embodiment, the lipid domain may be from a compound of formula III. In another embodiment, the lipid domain may be from a compound of formula IV. In another embodiment, the lipid domain may be from a compound of formula V. In another embodiment, the lipid domain may be from a compound of formula VI. Alterations of the hydrophilic portion of a lipid antigen have been described in, for example, Moody, et al., Science 278:283, which is incorporated herein in by reference in its entirety. A synthetic antigen according to the invention may be presented to a T-cell by an APC in association with CD1 molecules through the CD1 antigen presenting pathway or through the Class II MHC presenting pathway if conjugated to a protein carrier.

In another embodiment, the synthetic antigen is comprised of a hydrophilic domain from 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12 , NA-2-13, NA-2-14, NA-2-15, NA-2-16, or NA-2-17, and a hydrophobic domain not associated with said lipid portion in a naturally occurring lipid antigen found in wild-type Mycobacterium tuberculosis. Alternatively, in another embodiment, the hydrophilic domain may be from M-2, NA-2, or the total lipid fraction from M. tuberculosis. In another embodiment, the hydrophilic domain may be from a compound of formula I. In another embodiment, the hydrophilic domain may be from a compound of formula II. In another embodiment, the hydrophilic domain may be from a compound of formula III. In another embodiment, the hydrophilic domain may be from a compound of formula IV. In another embodiment, the hydrophilic domain may be from a compound of formula V. In another embodiment, the hydrophilic domain may be from a compound of formula VI.

The present invention may be used in mammals, including, but not limited to, humans, cats, dogs, horses, sheep, cattle, nonhuman primates, rodents, guinea pigs, etc. The present composition and methods are, therefore, intended for human as well as veterinarian use.

A vehicle is preferred, but not necessary to administer the antigens. The terms “pharmaceutically acceptable vehicle” or “vehicle” refer to any generally acceptable excipient or drug delivery composition that is relatively inert and non-toxic. Exemplary vehicles include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates such as lactose, sucrose, dextrose, mannose, albumin, starch, cellulose, silica gel, polyethylene glycol (PEG), dried skim milk, rice four, magnesium stearate, liposomes, and the like. Suitable formulations and additional vehicles are described in Remington's Pharmaceutical Sciences, 17^{th} Ed., Mack Pub. Co., Easton, Pa., the teachings of which are incorporated herein by reference in their entirety. Such preparations may be sterilized and, if desired, mixed with auxiliary agents, e.g., lubricants, preservatives, stabilizers, wetting agents, emulsifiers, slats for influencing osmotic pressure, buffers, coloring, and/or aromatic substances and the like which do not deleteriously react with the active compounds. They may also be combined where desired with other active substances, e.g., enzyme inhibitors, to reduce metabolic degradation.

Accordingly, in a third aspect, the present invention provides a variety of pharmaceutical compositions. Such pharmaceutical compositions comprise a therapeutically (or prophylactically) effective amount of a composition comprising at least one nonpeptide antigen isolated and purified from M. tuberculosis, and a vehicle. Preferably, the antigen is selected from an isolated and purified 2640-35A, 2640-38C, 2640-47E, 2640-47F, 2647-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, or NA-2-17 compound. In another embodiment, M-2, NA-2, or the total lipid fraction from M. tuberculosis may also be used as antigens. In another preferred embodiment of the invention, the composition further comprises at least one T-cell stimulating compound, which may either be an adjuvant or a protein carrier. The pharmaceutical composition, if desired, may also contain minor amounts of wetting or emulsifying agents, or pH buffering agents, or preservatives. Typical preservative may include potassium sorbate, sodium metabisulfite, methyl paraben, propyl paraben, thimerosal, etc.

The vaccine compositions of the present invention may also comprise other antigens, including peptide antigens or mixtures of lipid antigens, resulting in a multi-valent vaccine composition comprising one or more of the lipid antigens of the present invention.

The compositions of the present invention may be a liquid solution, emulsion, tablet, pill, capsule, sustained release formulation, or powder. The method of administration may dictate how the composition will be formulated. For example, the composition may be formulated as a suppository, with traditional binders and carriers such as triglycerides. Oral formulation may include standard vehicles such as pharmaceutical grades of mannitol, lactose, starch, magnesium stearate, sodium saccharine, cellulose, magnesium carbonate, etc.

In a preferred embodiment, the lipid antigens of the invention are formulated into liposomes and, optionally, with an adjuvant such as, but not limited to QS-21. The liposomes may comprise any known phospholipid, such as, but not limited to, 1,2-distearoyl-sn-glycero-3-phosphocholine (DSPC). In addition, cholesterol and D, L-erythro-6-glucose-corynomycolate and/or D, L-threo-6-glucose-corynomycolate may be added. Other carrier phospholipids include DLPC, DMPC; DPPC; DOPC; DMPE; DPPE; DOPE; DMPA.Na; DPPA.Na; DOPA.Na; DMPG.Na; DPPG.Na; DOPG.Na; DMPS.Na; DPPS.Na; DOPS.Na; DOPE-GLUTARYL.(Na); TETRA CARDIOLIPIN.(Na) MYRISTOYL; DPPE-mPEG-2000.Na; DPPE-mPEG-5000.Na D; DPEG-2000.Na PPE-CARBOXY and DOTAP.Cl. The ratio of carrier lipid to lipid antigen may range from 20:1 to 1:10. Cruder fractions of lipid antigens are preferably formulated with lesser amounts of carrier lipid, for example, from 2:1 to 1: 10, preferably from 2:1 to 1:2 lipid carrier to lipid antigen. More pure preparations of lipid antigens may be prepared using from 1:1 to 20:1, preferably 2:1 to 10:1 ratios of lipid carrier to lipid antigen. Other ratios according to the invention include, but are not limited to 3:1, 4:1, 5:1, 6:1, 7:1, 8:1, 9:1 or 10:1 lipid carrier to lipid antigen.

The ratio of phospholipid to cholesterol, if cholesterol is present, may range from 20:1 to 1:10. Other ratios of cholesterol to lipid carrier include, but are not limited to, from 2: 1 to 1:10, preferably from 2:1 to 1:1, from 1:1 to 20:1, preferably 2:1 to 10:1, 3:1, 4:1, 5:1, 6: 1, 7:1, 8:1, 9:1 or 10:1.

The liposome formulations of the invention are preferably prepared by sonicating or vortexing (i.e., vigorously shaking) a mixture of phospholipid carrier and lipid antigen. The mixture optionally includes cholesterol, and may also include D, L-erythro-6-glucose-corynomycolate and/or D, L-threo-6-glucose-corynomycolate. Most preferably, the mixture, either sonicated and/or vortexed or neither, is extruded through a filter membrane of defined pore size. The extrusion should be done at a temperature above the Tc (crystal transition temperature) of the lipids to be formulated. Preferably, the membrane pores are from 50 nm to 2000 nm, 100 nm to 1000 nm, 200 nm to 800 nm, or 400 nm to 800 nm. In preferred embodiments, the liposome formulation is extruded through a 400 nm filter membrane, or an 800 nm filter membrane. The extrusion is performed by any method known to one of skill in the art. Preferably, it is done using an extrusion device, for example, but not limited to, the Avanti Mini-Extruder, Catalog No. 610000 (Avanti Polar Lipids, Inc., Alabaster, Ala.). In a preferred embodiment, an Avanti Mini-Extruder is used according to the standard protocols provided by the manufacturer. Optionally, an adjuvant is added after extrusion. In a preferred embodiment, the lipid vesicles in the extruded vaccine composition have an average radius of from 50 nm to 500 nm, from 100 nm to 400 nm, from 100 nm to 300 nm, or from 100 nm to 200 nm. In other embodiments, the lipid vesicles in the extruded vaccine composition have a radius of less than 1000 nm, less than 750 nm, or less than 500 nm. In another embodiment, the lipid vesicles in the extruded vaccine composition also have a radius of greater than 10 nm, or greater than the theoretical limit of vesicles that can be formed by the lipid carrier being utilized.

The efficacy of any given vaccine formulation is readily determinable by one of skill in the art. For example, several animal models for M. tuberculosis are known, such as those detailed in McMurray, et al., 2000, Clin Infec Dis, Suppl 3:s210-2; Baldwin et al., 1998, Infect Immun, 66(6):2951-9; and McMurray et al., 1996, Curr Top Microbiol Immunol, 215:157-79. Preferred doses of lipid antigen are from 0.1 to 10 mg per dose; preferred doses of adjuvant are from 10 to 200 μg per dose.

The vehicle may be added to the composition at any convenient time. In the case of a lyophilized vaccine, the vehicle may, for example, be added immediately prior to administration. Alternatively, the final product may be manufactured with the vehicle.

The immunogenic vaccine compositions of the invention may be administered intravenously, parenterally, intramuscularly, subcutaneously, intradermally, orally, nasally, topically, by inhalation, by implant, by injection, or by suppository. The composition may be administered in a single dose or in more than one dose (e.g., boosted) over a period of time to confer the desired effect.

For enteral or mucosal application (including via oral and nasal mucosa), particularly suitable are tablets, liquids, drops, suppositories or capsules. A syrup, elixir or the like may be used wherein a sweetened vehicle is employed. Topical application may also be used for example, in intraocular administration. Alternative methods of administration may include an immune-stimulating complex (ISCOM) as described in U.S. Pat. No. 4,900,549. In addition to liposome, viral vectors, microspheres, and microcapsules are available and may be used.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

The method of administration will vary in accordance with the type of disorder and microorganism sought to be controlled or eradicated. The dosage of the vaccine will be dependent upon the amount of antigen, its level of antigenicity, and the route of administration.

Moreover, the actual effective amounts of compound or drug may vary according to the specific composition being utilized, the mode of administration and the age, weight and condition of the patient, for example. As used herein, an effective amount of the drug is an amount of the drug which elicits or boosts an immune response to the lipid antigen. Dosages for a particular patient may be determined by one of ordinary skill in the art using conventional considerations (e.g., by means of an appropriate, conventional pharmacological protocol).

A fourth aspect of the invention covers methods for eliciting, stimulating or enhancing an immune response in an animal to a M. tuberculosis antigen in a mammal comprising the step of administering to the mammal an effective amount of a vaccine composition comprising at least one nonpeptide antigen isolated from M. tuberculosis, which may also comprise a T-cell stimulating compound or compounds. These nonpeptide antigens may be isolated from a lipid extract of M. tuberculosis. Preferably these lipids are nonpeptide antigens associated with different polarity classes of lipids. These may be fractionated into different polarity classes by fractionation with solvents of different polarity on a silica gel column or similar means (i.e., fractionation into nonpolar classes with hexane or chloroform, intermediate polarity with acetone, and higher relative polarity with methanol). These different polarity classes may be further fractionated by normal phase or reversed phase HPLC to yield purified nonpeptide antigens. Preferably, the purified nonpeptide antigens described herein are selected from nonpolar, intermediate polarity, and high polarity classes. More preferably, the purified nonpeptide antigens are 2640-35A, 2640-38C, 2640-47E, 2640-47F, 2640-47H, 2640-48D. One preferred embodiment of the inventive method covers administering to a mammal a vaccine composition further comprising at least one T-cell stimulating compound. The T-cell stimulating compound may be either an adjuvant or a protein carrier. The composition may consist of conjugation of the nonpeptide antigen to a protein carrier and one or more adjuvants. Alternatively, in yet another preferred embodiment, the adjuvant used in the method described herein is preferably selected from a mineral salt adjuvant, Incomplete or Complete Freund's Adjuvant, Bacille Calmette-Guerin adjuvant, block polymer adjuvant, cholera toxin, cytokines, CpG motif-containing adjuvant, oil/water emulsion adjuvant, MF-59 adjuvant, LeIF adjuvant, liposome adjuvant, ISCOM adjuvant, Monophosphoryl lipid A adjuvant, biodegradable microsphere adjuvant, muramyl dipeptide adjuvant, polyphosphazene adjuvant, and a saponin adjuvant. In another embodiment of this method, the composition administered may include one or more adjuvants.

Various publications are cited herein, the disclosures of which are incorporated by reference in their entireties.

The invention being thus described, it will be obvious that the same may be varied in many ways. Such variation are not to be regarded as a departure from the spirit and scope of the invention, and all such modifications, as would be obvious to a person skilled in the art, are intended to be included in the scope of the following claims.

The following examples are meant to be illustrative and not limiting in any way.

### EXAMPLES

### Example 1

Purification of Lipid Antigens

Materials and Methods

All solvents were HPLC grade, purchased from VWR (Boston, Mass.). HPLC columns were purchased from Varian (Walnut Creek, Calif.). M. tuberculosis strain H37Rv was purchased from Colorado State University as irradiated whole cells, propagated in GAS media at 37° C. for two weeks, and shipped frozen. Silica gel (Aldrich, Milwaukee, Wis.) (35-70 mesh, 40 Å) was used for column chromatography. Electrospray ionization (ESI) mass spectra were measured at Mass Consortium (San Diego, Calif.) and Jing Hong Custom NMR Services (Cambridge, Mass.). FAB and EI mass spectra were measured on VG Analytical ZAB 2-SE high field mass spectrometer at M-Scan Company (West Chester, Pa.). NMR spectra were recorded on an Inova-500 NMR instrument at Boston College (Chestnut Hill, Mass.) and a Varian-400 NMR Instrument at Jing Hong Custom NMR Services (Cambridge, Mass.). Elemental analysis was performed at Quantitative Technologies Inc. (Whitehouse, N.J.).

Semi-preparative HPLC condition:

Column: Microsorb Si, 10.0×250 mm, 5 μm.

Flow rate: 3 ml/min.

Mobile phase I (for the acetone fraction): linear gradient

| Time (min.) | CHCl₃ | MeOH | H₂O |
|---|---|---|---|
| 0 | 100 | 0 | 0 |
| 5 | 89 | 10 | 1 |
| 15 | 78 | 20 | 2 |
| 35 | 65 | 30 | 5 |
| 45 | 65 | 30 | 5 |
| 55 | 78 | 20 | 2 |
| 60 | 100 | 0 | 0 |

Mobile phase II (for the chloroform fraction): linear gradient

| Time (min.) | CHCl₃ | Hexane |
|---|---|---|
| 0 | 60 | 40 |
| 30 | 90 | 10 |
| 40 | 100 | 0 |

Mobile phase III (for the methanol fraction): linear gradient

| Time (min.) | CHCl₃ | MeOH | H₂O |
|---|---|---|---|
| 0 | 72 | 25 | 3 |
| 20 | 65 | 30 | 5 |
| 30 | 60 | 35 | 5 |
| 55 | 60 | 35 | 5 |
| 60 | 72 | 25 | 3 |

Analytical HPLC Conditions

Column: Microsorb Si, 4.6×250 mm, 5 μm.

Flow rate: 1 ml/min.

Detector: PL-ELS 1000

Mobile phase IV (for lipids isolated from the acetone fraction): linear gradient

| Time (min.) | CHCl₃ | MeOH | H₂O |
|---|---|---|---|
| 0 | 100 | 0 | 0 |
| 5 | 89 | 10 | 1 |
| 10 | 78 | 20 | 2 |
| 15 | 78 | 20 | 2 |
| 20 | 65 | 30 | 5 |
| 25 | 78 | 20 | 2 |
| 30 | 100 | 0 | 0 |

Mobile phase V (for lipids isolated from the chloroform fraction): linear gradient

| Time (min.) | CHCl₃ | Hexane |
|---|---|---|
| 0 | 60 | 40 |
| 15 | 90 | 10 |
| 20 | 100 | 0 |

Isolation of lipid fraction from M. tuberculosis H37Rv

The mixture of 50 g wet pellet of M. tuberculosis H37Rv irradiated whole cells) in 1 L of CHCl₃/MeOH (2:1) was shaken for 4 h on a plate shaker at room temperature. The mixture was transferred to 25 centrifuge tubes and spun at 2000 g for 10 min. at 20° C. The supernatant was removed and the pellet was re-extracted with 1 L of CHCl₃/MeOH (1:1) overnight at room temperature as above. After the supernatant was removed, the pellet was re-extracted with 1 L of CHCl₃/MeOH (1:2) for 4 h by the same method. The supernatant was combined and the solvents were evaporated under vacuum at 20° C. to afford 1.9 g of total lipids as an orange oil.

Crude fractionation of the lipid fraction

About 1.5 g of the lipid fraction were suspended in hexane and then subjected to a short glass column packed with 30 g of silica gel. The column was washed successively with 150 ml of hexane, 250 ml of CHCl₃, 250 ml of acetone, and 250 ml of MeOH to afford four different solvent fractions (polarity classes of lipids). The CHCl₃ fraction yielded 0.21 g of a yellow semi-solid. The acetone fraction yielded 0.47 g of a dark red syrup. The MeOH fraction yielded 0.65 g of an orange syrup.

Purification of the acetone fraction by silica gel column chromatography

The above acetone fraction (0.45 g) was dissolved in a small amount of CHCl₃ and then applied to a glass column (1.2×50 cm) packed with 15 g of silica gel. The column was eluted with 100 ml of 3% MeOH in CHCl₃, followed by 50 ml of 10% MeOH in CHCl₃, and finally with 100 ml of 50% MeOH in CHCl₃. The eluate was collected into glass tubes containing 10 ml per fraction. The fractions were checked by thin layer chromatography (TLC) and combined according to TLC results to afford 6 fractions: Fraction A (47 mg, yellow), Fraction B (33 mg, yellow), Fraction C (136 mg, dark red), Fraction D (13 mg, orange), Fraction E (105 mg, brown), and Fraction F (15 mg, brown).

Purification of Fraction E (Acetone fraction) by semi-preparative HPLC

The above Fraction E from the crude acetone fraction was further purified by normal phase silica semi-preparative HPLC using mobile phase I and the column indicated above. A total of three runs were carried out, 20 mg of Fraction E was injected in each run and the fractions were collected at fraction/min. Two purified compounds were isolated. Thirty-four mg of compound 2640-35A was obtained after the solvents were evaporated under vacuum. A total of 2.7 mg of compound 2640-38C was obtained.

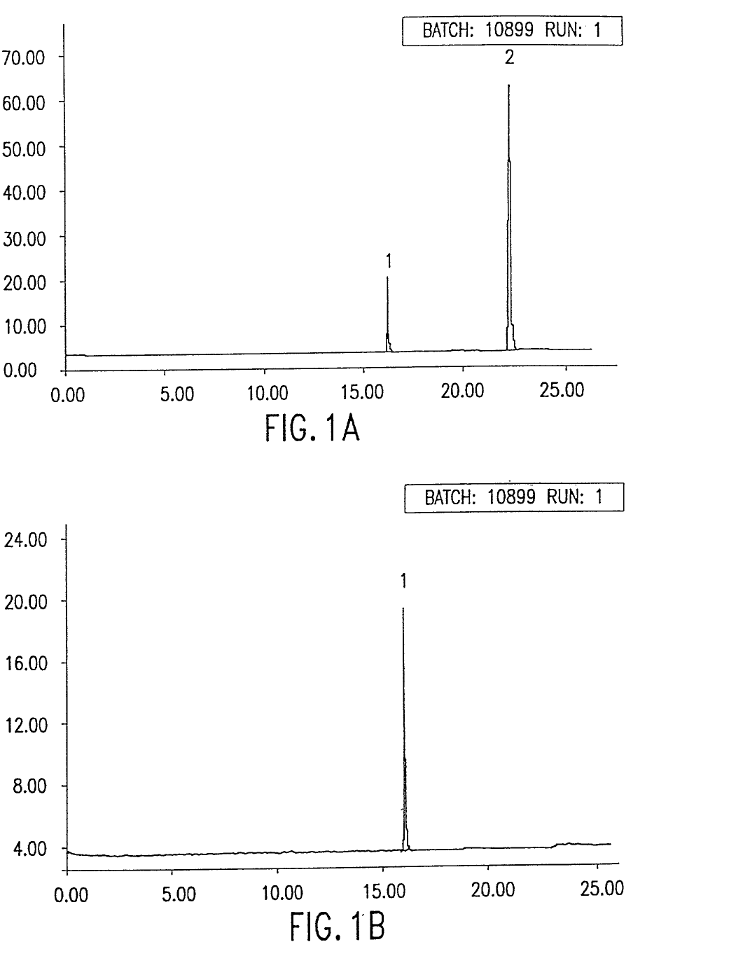

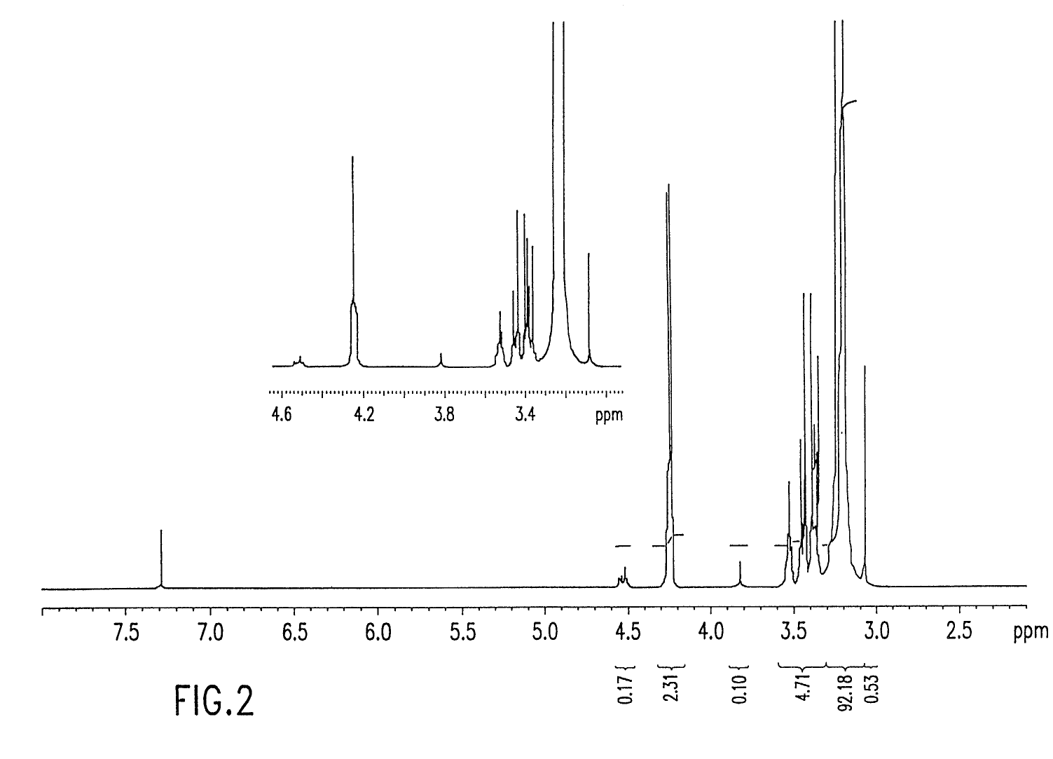

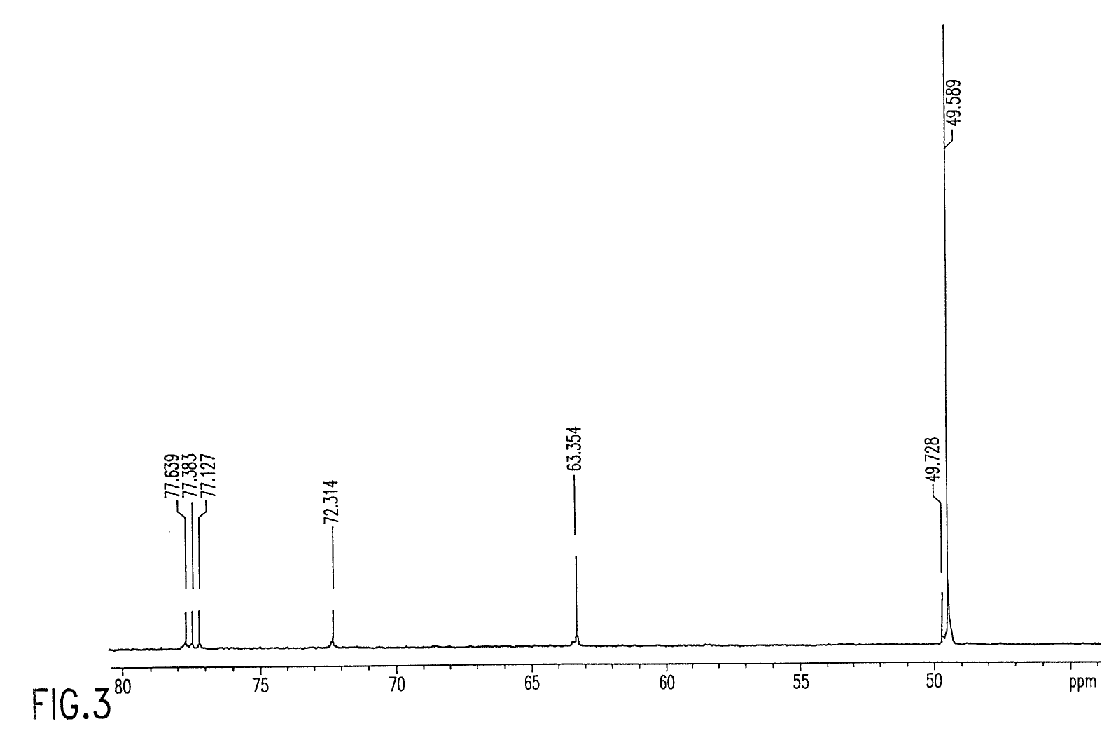

Compound 2640-35A was a colorless oil. It was soluble in a mixture of chloroform and methanol, but poorly soluble in individual solvents. On TLC, it stained positively with Bial's orcinol reagent (greenish yellow), weakly positive in ninhydrin, negative with molybedenum blue, and negative in 10% sulfuric acid/ethanol. An analytic HPLC profile is shown in FIG. 1. Positive ion ESI mass spectral analysis yielded an apparent ion at m/z 686 and negative ion ESI-MS gave two ions at m/z 1278, 1250. Negative ion FAB-MS showed two major ions at 1277, and 1249. El-MS showed fragment ions at m/z 61, 648, 663, and 679. ¹H-NMR (500 MHz, CDCl₃/CD₃OD 2:1) of 2640-35A is shown in FIG. 2. ¹³C-NMR (125 MHz, CDCl₃/CD₃OD 2:1) is shown in FIG. 3. Elemental analysis showed C: 38.9%, H: 8.7%, O: 51.7%, and N: <0.05%.

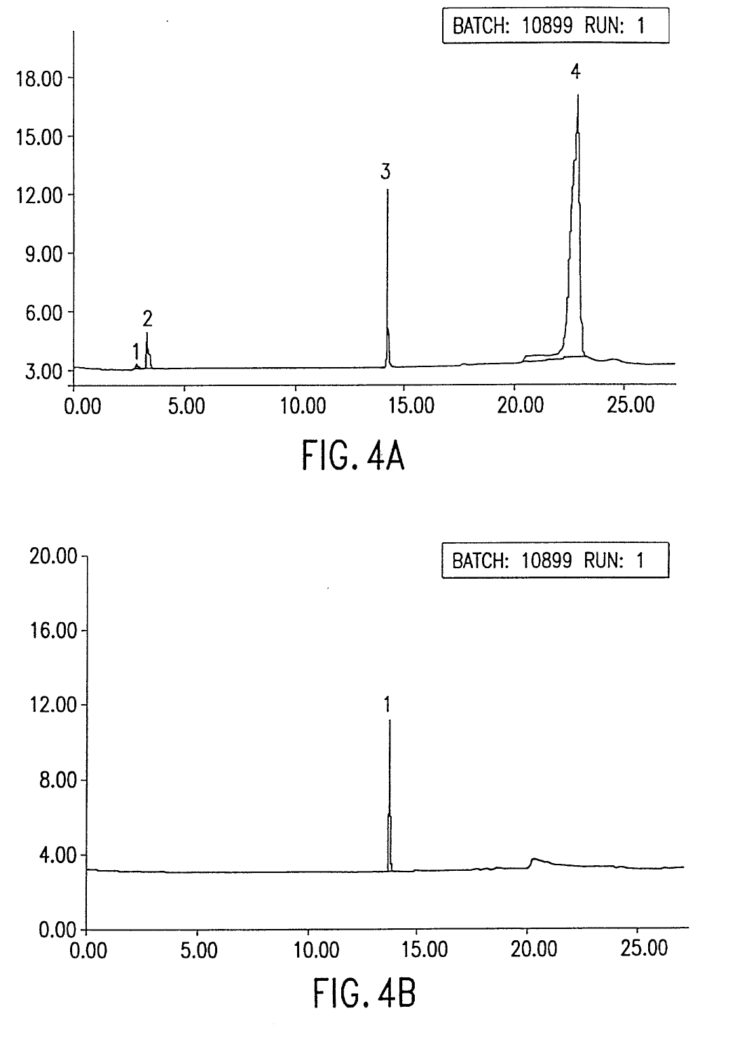

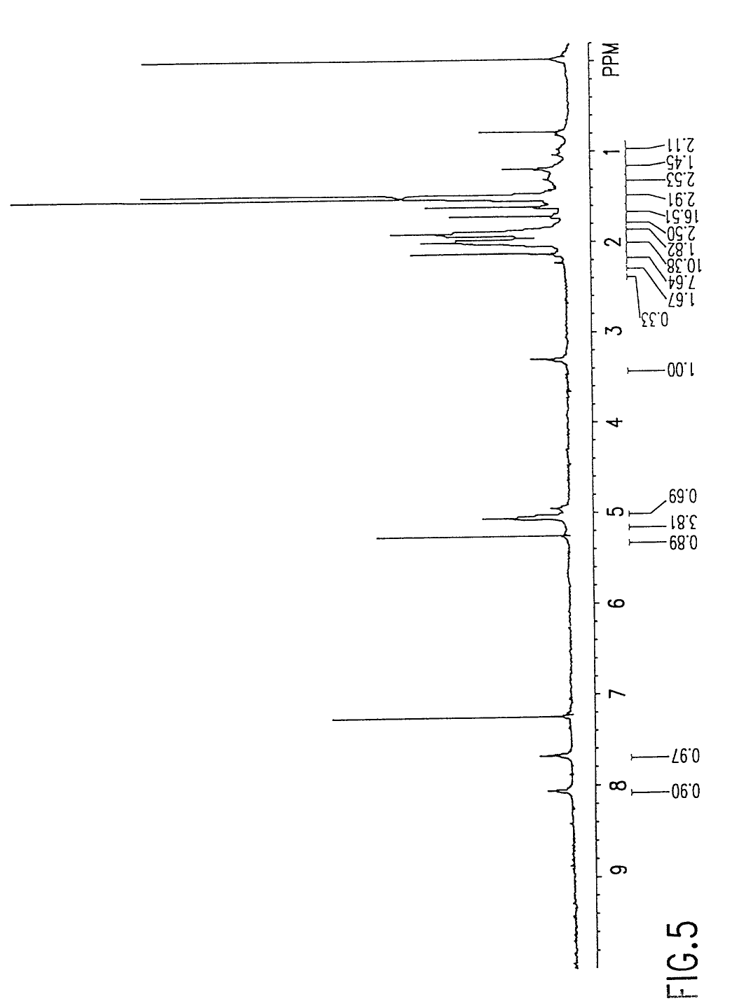

Compound 2640-38C was a white semi-solid. It was soluble in methanol and insoluble in chloroform and acetone. On TLC, it was positive in Bial's orcinol reagent (blue), weakly positive in ninhydrin, positive in 10% H₂ SO₄/EtOH, and negative in molybedenum blue. An analytic HPLC profile using mobile phase IV and the column indicated above is shown in FIG. 4. Negative ion ESI mass spectral analysis yielded an apparent ion at m/z 1414.

Purification of the chloroform fraction by silica gel chromatography

The above chloroform fraction (0.20 g) was dissolved in a small amount of a mixture of hexane/CHCl₃ and then was applied to a glass column (1.2×50 cm) packed with 10 g of silica gel. The column was eluted with 50 ml of hexane/CHCl₃ (5:1), followed by 100 ml of hexane/CHCl₃ (4:1), 50 ml of hexane/CHCl₃ (1:4), 50 ml of hexane/CHCl₃ (1:10), and finally with 100 ml of CHCl₃. The eluate was collected into glass tubes at 8 ml per fraction. The fractions were pooled according to TLC patterns to afford 6 fractions: Fraction A (6 mg, white), Fraction B (13 mg, yellow), Fraction C (13 mg, yellow), Fraction D (33 mg, yellow), Fraction E (37 mg, yellow), and Fraction F (27 mg, yellow).

Purification of Fraction D (Chloroform fraction) by semi-preparative HPLC

The above Fraction D was further purified by normal phase silica semi-preparative HPLC using mobile phase II and the column indicated above. Total 30 mg of Fraction D was injected and the fractions were collected at fraction/30 sec. The solvent of Fraction 27 was evaporated under vacuum to afford <0.1 mg of compound 2640-47E as a light yellow oil. The combination of fractions 28-31 afforded 1.9 mg of 2640-47F as a yellow oil. The combination of fractions 33 and 34 afforded 1.9 mg of 2640-47H as a light yellow oil. The combination of fractions 54 and 56 afforded 0.6 mg of compound 2640-48D as a colorless oil.

Compound 2640-47E was a light yellow oil. It was soluble in CHCl₃. On TLC, it was positive in Bial's orcinol reagent, positive in 10% H₂ SO₄/EtOH, negative in ninhydrin, and negative in molybedenum blue. On analytic HPLC using mobile phase IV and the column indicated above, it had a retention time of 9.9 min.

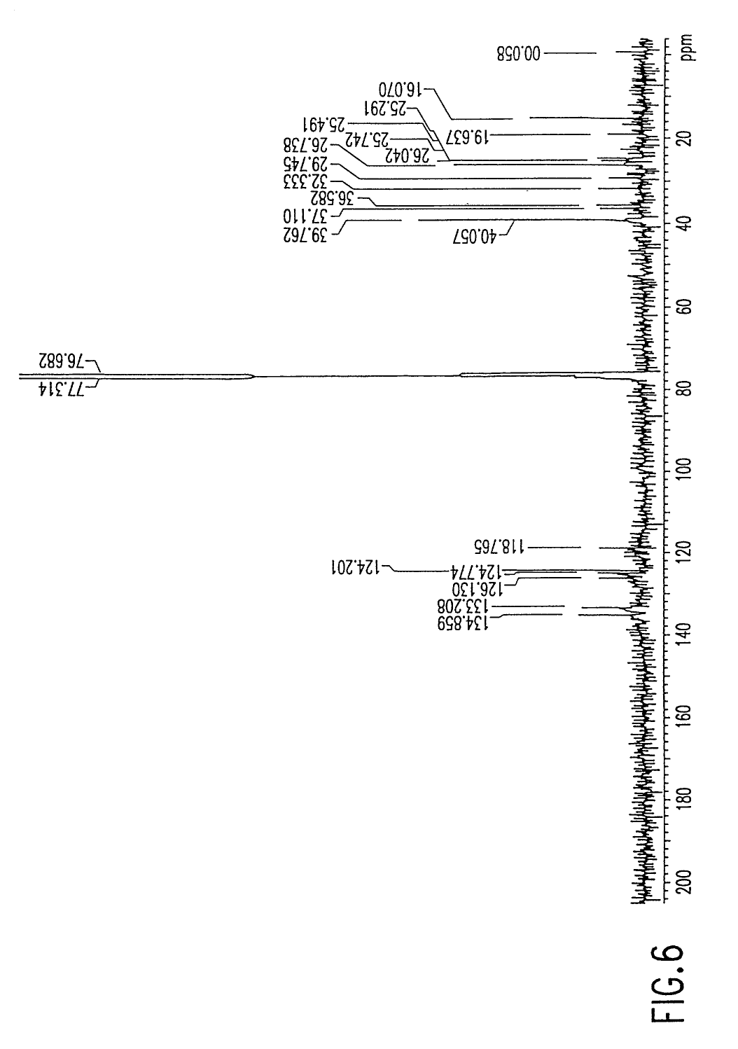

Compound 2640-47F was a yellow oil. It was shown to be soluble in CHCl₃. On TLC, it was shown to be positive in Bial's orcinol reagent, positive in 10% H₂SO₄/EtOH, negative in ninhydrin, and negative in molybdenum blue. On analytic HPLC using mobile phase IV and the column indicated above, it had a retention time of 10.9 min. On positive EIS mass spectral analysis, it yielded an apparent ion at m/z 851. ¹H-NMR (400 MHz, CDCl₃) is shown in FIG. 5. ¹³C-NMR (100 MHz, CDCl₃) is shown in FIG. 6. Compound 2640-47H is a light yellow oil. It is soluble in CHCl₃. On TLC, it is positive in Bial's orcinol reagent, positive in 10% H₂SO₄/EtOH, negative in ninhydrin, and negative in molybedenum blue. On analytic HPLC using mobile phase IV and the column indicated above, it had a retention time of 11.9 min.

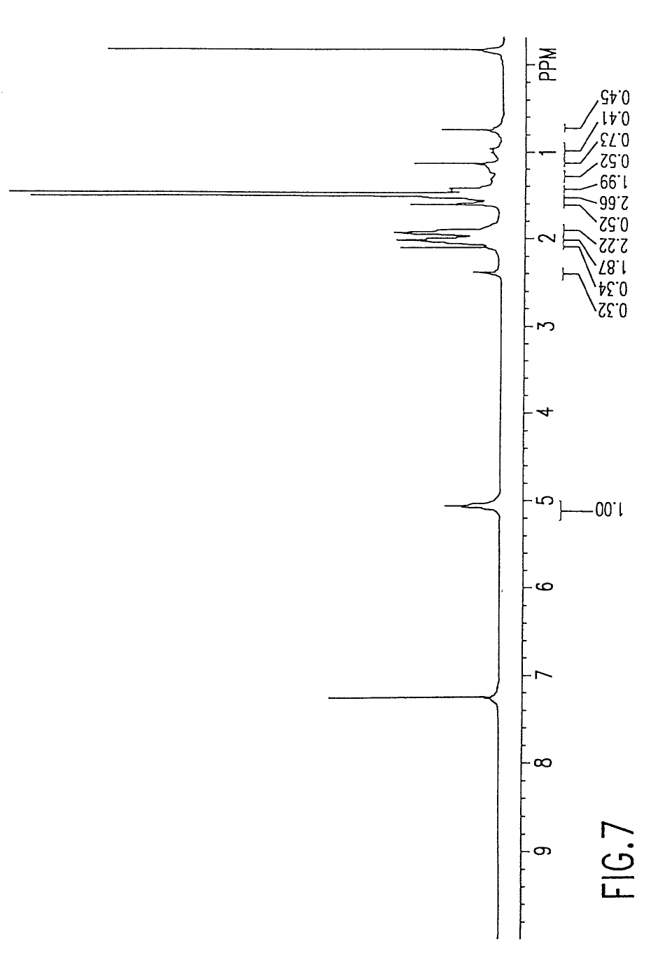

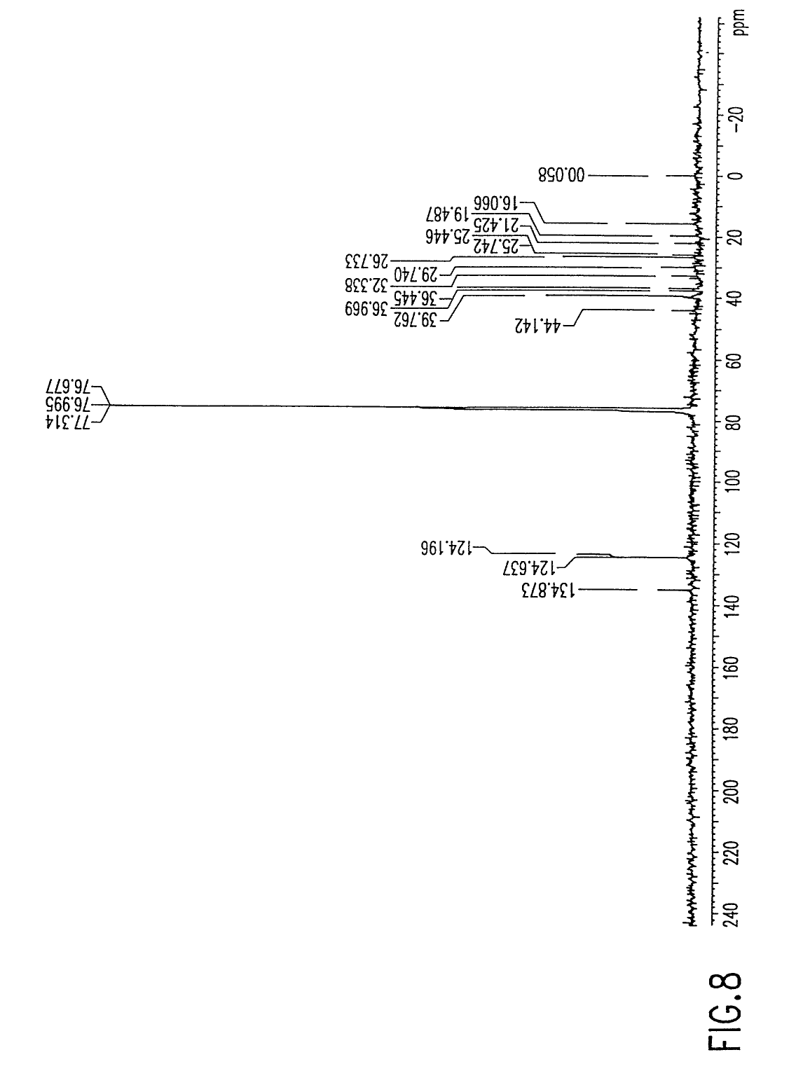

Compound 2640-48D was a colorless oil. It was soluble in CHCl₃. On TLC staining, it was positive in Bial's orcinol reagent, positive in 10% H₂SO₄/EtOH, weakly positive in ninhydrin, and negative in molybedenum blue. On analytic HPLC using mobile phase IV and the column indicated above, it had a retention time of 20.4 min. Positive ion ESI mass spectral analysis showed an apparent ion at m/z 663. ¹H-NMR (400 MHz, CDCl₃) is shown in FIG. 7. ¹³C-NMR (100 MHz, CDCl₃) is shown in FIG. 8.

Acetone precipitation of the methanol fraction

The methanol fraction (0.50 g) was dissolved in small amount of CHCl₃/MeOH (2:1), then an excess of acetone was added. The precipitate was filtered to afford two fractions, acetone soluble fraction (M-1, 350 mg) and acetone insoluble fraction (M-2, 72 mg).

Purification of M-2 (acetone insoluble fraction) by semi-preparative HPLC

M-2 (30 mg) was fractionated by semi-preparative HPLC using mobile phase V and the column indicated above. The elute was collected by glass test tubes at 1 minute per fraction rate. Total 60 fractions were collected and these fractions were combined as follows. Fr. 4-6 (2.9 mg, M-2-1); Fr. 7-10 (2.9 mg, M-2-2); Fr. 11-14 (4.0 mg, M-2-3); Fr. 15-17 (2.5 mg, M-2-4); Fr. 18-24 (6.8 mg, M-2-5); Fr. 25-27 (1.3 mg, M-2-6); Fr. 28-31 (1.8 mg, M-2-7); Fr. 32-35 (1.4 mg, M-2-8); Fr. 36-37 (2.2 mg, M-2-9); Fr. 38-41 (3.5 mg, M-2-10); Fr. 42-50 (1.2 mg, M-2-11); Fr. 51-600.7 mg, (M-2-12).

### Example 2

Immunoassay

The immunogenicity of nonpeptide antigens disclosed are determined in an in vitro cell proliferation assay, using T cell lines or PBMC from individuals infected with M. tuberculosis. T cells (5×10⁴) in 96-well plates are cultured in RPM 1640 (with 10% fetal calf serum) with or without nonpeptide antigen in the presence of irradiated GM-CSF/IL-4 treated monocytes (5×10⁴) as APCs. On day 3 of culture, ³H-thymidine (1 uCi/well) is added and incorporation determined 6 hours after thymidine addition. The uptake of thymidine in media-stimulated cells is calculated as the background subtracted from the uptake of thymidine in antigen-stimulated cells to determine antigen-specific proliferation. Nonpeptide antigens that are recognized by T cells from M. tuberculosis-infected individuals cause an increase in proliferation above that caused by media alone.

The nonpeptide antigens and vaccine compositions disclosed are also used to immunize mammals by a variety of routes. For example, guinea pigs are immunized by subcutaneous, intramuscular, intranasal, intraperitoneal, or oral route. One or more booster immunizations are given. The immune response to M. tuberculosis is evaluated as an increase in serum antibody response. This is determined in a standard enzyme immunoassay against killed Mycobacteria or Mycobacteria extract.

The immune response is also measured by antigen-specific proliferative responses in splenic T-cells. Splenocytes or lymphocytes from immunized guinea pigs (Hartley or Strain II) are stimulated in culture with antigen such as killed mycobacteria, mycobacteria extract, or purified immunizing antigens. An immune response is demonstrated by an increased uptake of ³H-thymidine during the antigen stimulation. In one example, spleens are collected from guinea pigs immunized one or more times with the lipid compositions of the instant invention. Single cell suspensions of splenocytes are obtained from minced spleens treated with collagenase D. Nonadherent T-cells are obtained by passage through nylon wool. Adherent antigen presenting cells are collected for use as antigen presenting cells. T-cells (4×10⁵ cells/ml), irradiated-autologous antigen presenting cells (2×10⁵ cells/ml), and stimulating lipid antigen are incubated together in RPMI 1640 medium with 10% fetal bovine serum at 37° C. for three days. The culture is then pulsed with ³H-thymidine. The uptake of thymidine in media-stimulated cells is calculated as the background subtracted from the uptake of thymidine in antigen-stimulated cells to determine antigen-specific proliferation.

The immune response is also demonstrated by increased cytolytic response against infected cells such as macrophages. It is also demonstrated by increased cytokine production from antigen stimulated lymphocytes.

### Example 3

Purification of Lipid Antigens

Crude organic extract from M. tuberculosis H37Rv

Wet cell pellet (65.0 g) of M. tuberculosis H37Rv was extracted with hexane/methanol/water (500 ml/300 ml/30 ml) at room temperature for 2 hrs. After hexane phase was removed, 600 ml chloroform was added and the mixture was shaken continuously for several hours on an orbital shaker. The organic phase was separated, dried by rotary evaporation at 20-30° C. under vacuum, and finally dried by lyophilizing to afford 1.48 g crude organic extract (2.3%).

Acetone precipitation of above crude organic extract

Above organic extract (0.28 g) was dissolved in 1.5 ml of chloroform/methanol (2:1), then 30 ml of acetone was added. The precipitate was separated by centrifugation to afford two fractions, NA-1 (acetone soluble fraction) and NA-2 (acetone insoluble fraction).

Purification of NA-2 by semi-preparative HPLC

Above acetone insoluble fraction (NA-2, 40 mg) was purified by normal phase silica semi-preparative HPLC by using mobile phase VI and the column indicated. The eluate was collected by glass test tubes at 1 minute per fraction rate. Total 90 fractions were collected and the fractions were combined as follows according to their TLC pattern. Fr. 2-4 (6.0 mg, NA-2-1); Fr. 5 (2.3 mg, NA-2-2); Fr. 6 (0.5 mg, NA-2-3); Fr. 7-11 (1.3 mg, NA-2-4); Fr. 12-16 (1.7 mg, NA-2-5); Fr. 17-19 (2.1 mg, NA-2-6); Fr. 20-23 (2.2 mg, NA-2-7); Fr. 24-28 (1.9 mg, NA-2-8); Fr. 29-33 (lost); Fr. 34-37 (2.0 mg, NA-2-9); Fr. 38-41 (2.0 mg, NA-2-10); Fr. 42-43 (2.1 mg, NA-2-11); Fr. 44-51 (4.6 mg, NA-2-12); Fr. 52-59 (3.4 mg, NA-2-13); Fr. 60-69 (1.9 mg, NA-2-14); Fr. 70-71 (1.4 mg, NA-2-15); Fr. 72-77 (3.0 mg, NA-2-16); Fr.78-81 (5.3 mg, NA-2-17).

Purification of NA-2-12 by TLC

NA-2-12 (3.0 mg) was further purified by TLC (two 20×20 cm, 0.2 mm thick aluminium sheets, CHCl₃/MeOH/H₂O 60:20:2) to afford 1.5 mg NA-2-12-1 as yellowish solid.

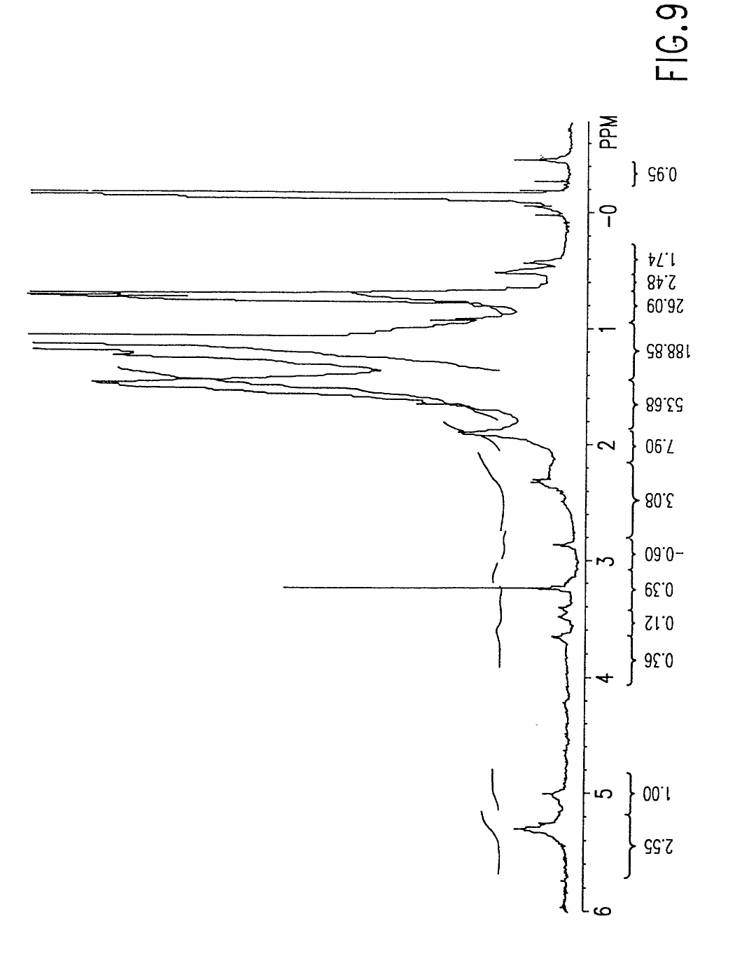

Compound NA-2-2 was a white solid. It was soluble in chloroform. On TLC, it was positive in Bial's orcinol reagent and negative in molybdenum blue. On analytic TLC, Its R_{f} value is 0.16 (CHCl₃/MeOH/H₂O 90:10:1) and 0.33 (CHCl₃/MeOH/H₂O 60:10:2). It had a retention time of 3.5 min. on analytic HPLC (mobile phase VII, using column indicated). Negative ESI mass spectrum showed two major ions at m/z 708 and 700. Its ¹H-NMR (400 MHz, CDCl₃) is shown in FIG. 9.

Compound NA-2-7 was a white solid. It was soluble in chloroform. On TLC, it was positive in Bial's orcinol reagent and negative in molybdenum blue. On analytical TLC, it had R_{f} value of 0.17 (CHCl₃/MeOH/H₂O 60:10:2). Its retention time on analytic HPLC is 12.8 min. (mobile phase VII, using column indicated). Negative ESI-MS yielded two apparent ions at m/z 708 and 700.

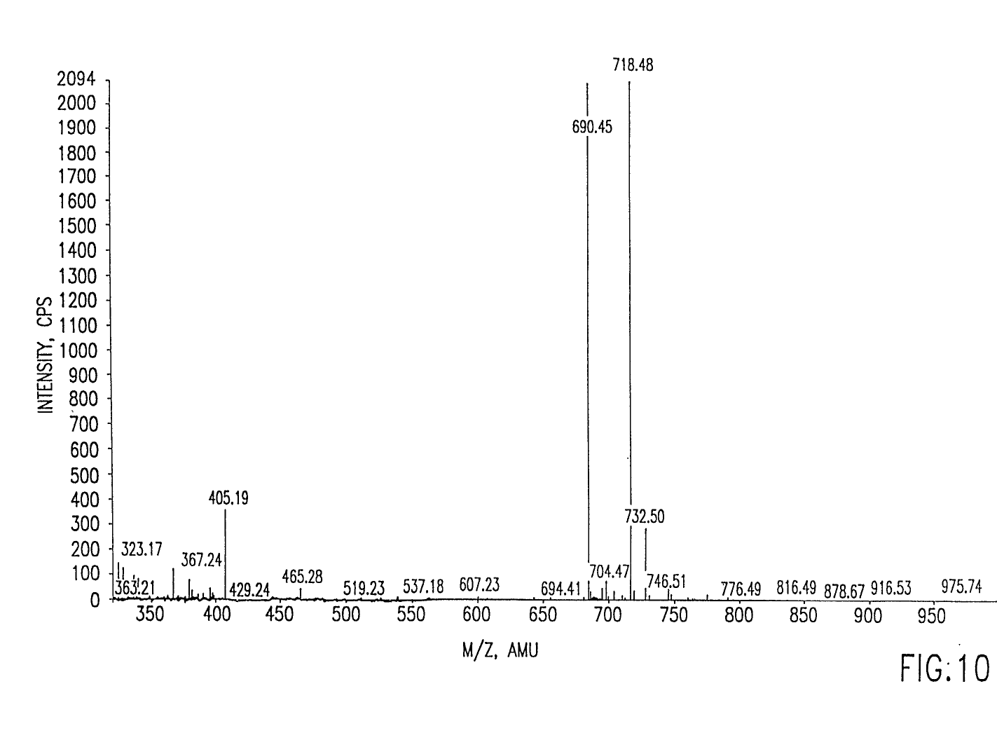

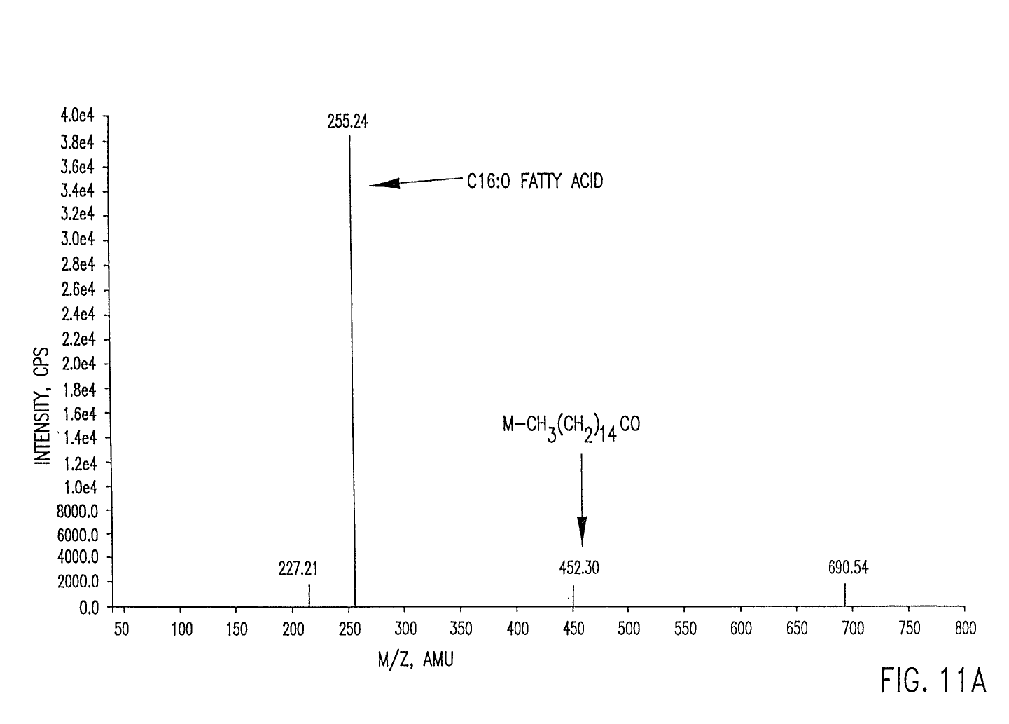

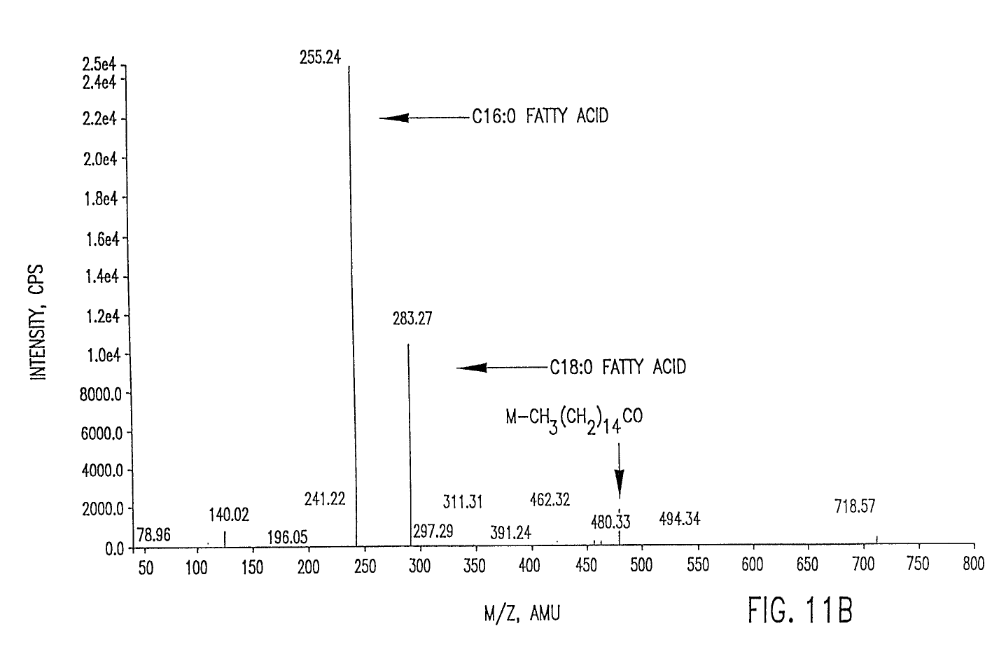

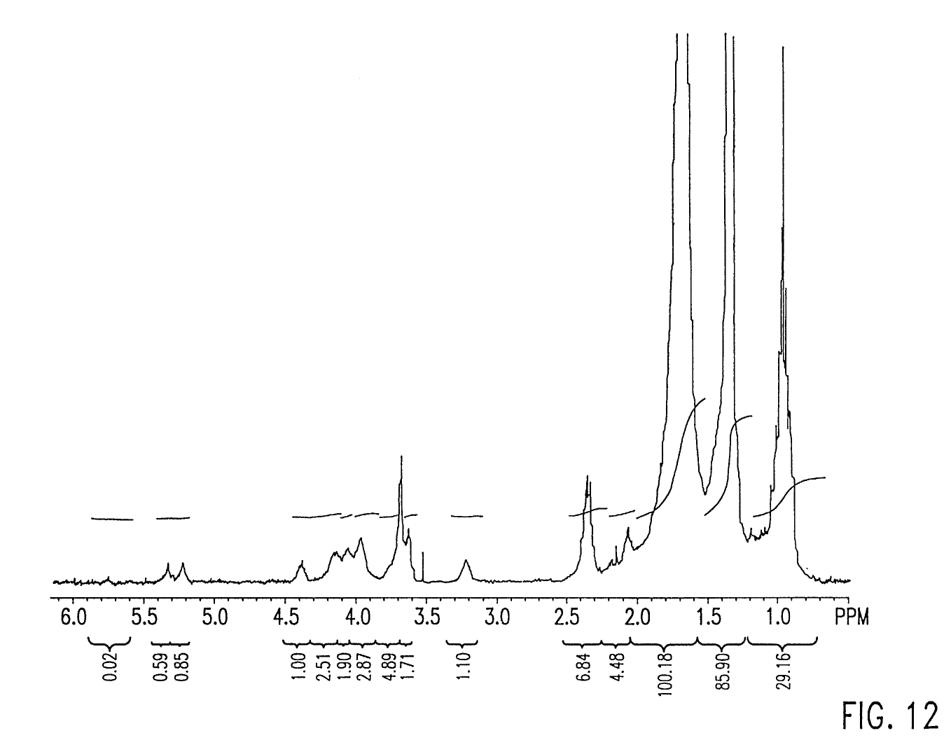

Compound NA-2-12-1 is a yellowish solid. It was soluble in chloroform. On TLC, it was negative in Bial's orcinol reagent, positive in molybdenum blue, negative in Dragendorff's reagent, and negative in Ninhydrin. On analytic TLC, it showed R_{f} value of 0.44 (CHCl₃/MeOH/H₂O 60:20:2). Its negative ESI-MS and negative ESI-MS/MS are shown in FIG. 10 and FIG. 11 respectively. Its ¹H-NMR (400 MHz, CDCl₃) is shown in FIG. 12. NA-2-12-1 is a mixture of three phospholipids, and their chemical structures were elucidated based on their ESI-MS, ESI-MS/MS, and ¹H-NMR spectra. The structure for [M-H]⁻ at m/z 690 is 1-palmitoyl-2-tuberculostearoyl-glycero-3-phosphate or 1-tuberculostearoyl-2-palmitoyl-glycero-3-phosphate and the structures for [M-H]⁻ at m/z 718 are 1palmitoyl-2-(2′-methyl)arachidoyl-glycero-3-phosphate or 1-(2′-methyl)arachidoyl-2-palmitoyl-glycero-3-phosphate and 1-stearoyl-2-tuberculostearoyl-glycero-3-phosphate or 1-tuberculostearoyl-2-stearoyl-glycero-3 -phosphate.

HPLC mobile phase VI: linear gradient

Column: Microsorb Si, 10.0×250 mm, 5 μm

Flow rate: 3 ml/min.

| Time (min.) | CHCl₃ | MeOH | H₂O |
|---|---|---|---|
| 0 | 90 | 9 | 1 |
| 60 | 73 | 24 | 3 |
| 70 | 60 | 35 | 5 |
| 90 | 60 | 35 | 5 |
| 100 | 90 | 9 | 1 |

HPLC Mobile phase VII: linear gradient

Column: Microsorb Si, 4.6×250 mm, 5 μm

Flow rate: 1/min.

| Time (min.) | CHCl₃ | MeOH | H₂O |
|---|---|---|---|
| 0 | 90 | 9 | 1 |
| 30 | 73 | 24 | 3 |
| 35 | 60 | 35 | 5 |
| 45 | 60 | 35 | 5 |
| 50 | 90 | 9 | 1 |

### Example 4

Proliferation Bioassays

M. tuberculosis infected subjects had the following features: 1) a history of clinical exposure to active tuberculosis, 2) a documented positive intradermal tuberculin (PPD) test (>15 mm in duration, WHO criteria) within one month prior to entry into the study, and 3) negative serology for human immunodeficiency virus.

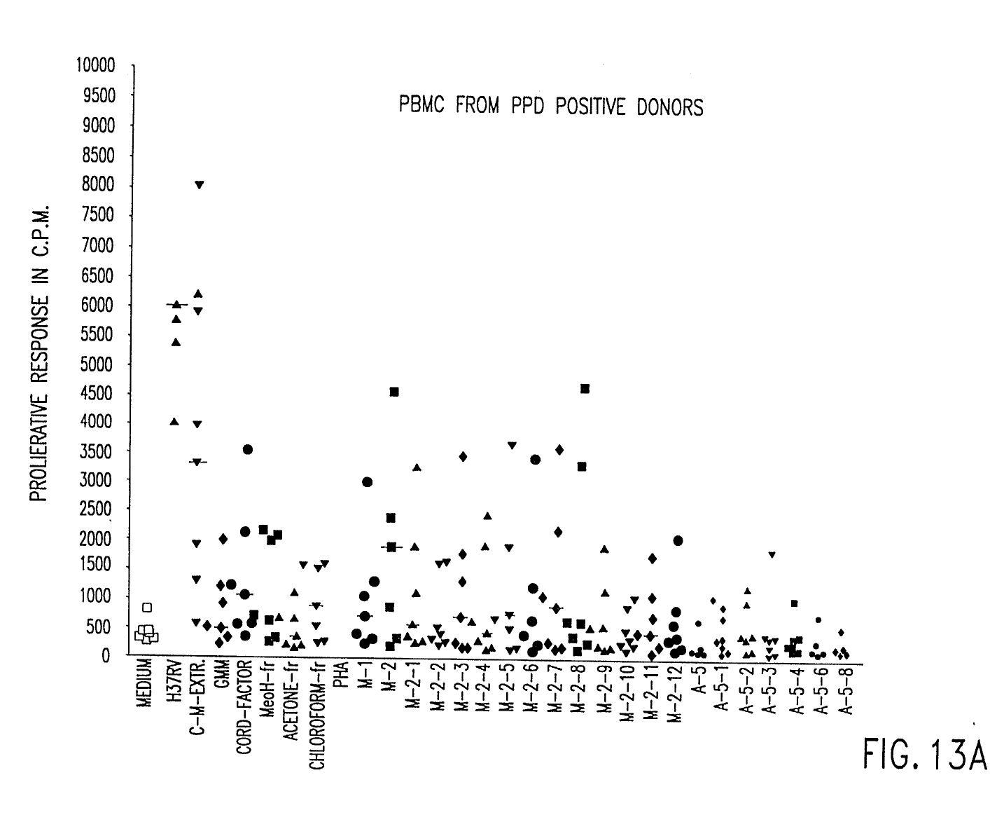

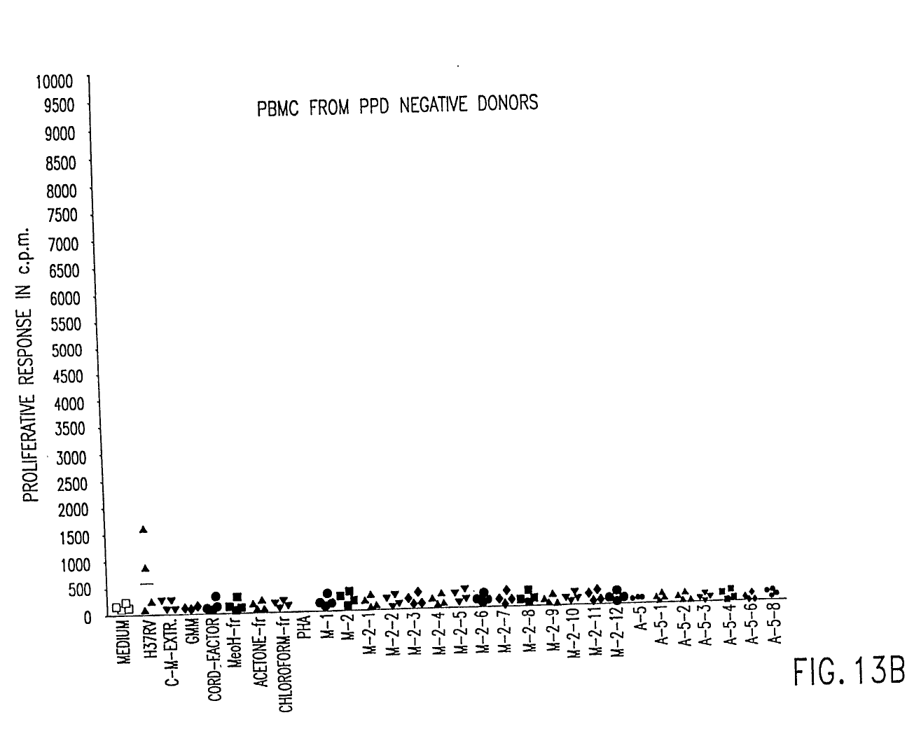

M. tuberculosis infected subjects were phlebotomized prior to INH therapy and responses of their PBL to antigen were compared to PBL obtained from healthy age and sex matched PPD negative controls with no history of M. tuberculosis exposure. Isolation of fresh PBL and monocytes was done by ficoll-hypaque gradient centrifugation, followed by plastic adherence for one hour. The nonadherent cells (PBL, lymphocytes) were collected and cryopreserved. The adherent cells (monocytes) were induced to differentiate into dendritic cells by culturing in medium containing GM-CSF (300 U/ml) and IL-4 (200 U/ml) for three days. The DCs were then harvested and irradiated (5000R) for use as APCs. All samples were processed on the day of phlebotomy, and isolated PBL were cryopreserved for 3 days to allow the differentiation of monocytes from each donor into dendritic cells. The PBL were then thawed and cultured with autologous dendritic cells in wells of 96 well flat bottom microtiter plates (100,000 PBL plus 50,000 DCs). Proliferation of the PBL samples in the presence of autologous dendritic cells was measured by ³H-thymidine incorporation with or without addition of lipid antigen containing fractions. Medium used for cultures was RPMI-1640 plus 10% FCS, 50 μM 2-ME, and 10 mM hepes. Results were reported as cpm of thymidine incorporation (see FIG. 13).

### Example 5

Vesicle Formulation of Lipid Antigens

Material and Methods

All solvents were HPLC grade, purchased from VWR (Boston, Mass.). Mycobacterium tuberculosis strain H37Rv was purchased from Colorado State University as irradiated whole cells, propagated in GAS media at 37° C. for two weeks and shipped frozen. DSPC and cholesterol were purchased from Avanti Polar Lipids, Inc. (Alabaster, Ala.). D, L-Erythro- and D, L-threo-6-glucose-corynomycolates were purchased from Matreya, Inc. (Pleasant Gap, Pa.).

Total lipid antigens isolated from M. tuberculosis H37Rv

The mixture of 50 g of wet cell pellet of M. tuberculosis H37Rv (gamma irradiated whole cells) in 1 L of CHCl₃/MeOH (2:1) was shaken for 4 h on an orbital shaker at room temperature. The mixture was transferred to 25 centrifuge tubes and spun at 2000 g for 10 min. at 20° C. The supernatant was removed and the pellet was reextracted with 1 L of CHCl₃/MeOH (1:1) overnight at room temperature as above. After the supernatant was removed, the pellet was reextracted with 1 L of CHCl₃/MeOH (1:2) for 4 h by the same method. The supernatants were combined, dried by rotary evaporation at 20-30° C. under vacuum, and finally dried by lyophilizing to afford 1.9 g of total lipids as an orange semisolid.

Vaccine formulation of total lipid antigens from M. tuberculosis H37Rv.

The mixture of 1.4 ml of above total lipids in CHCl₃/MeOH (1:1) solution (14 mg, 10 mg/ml), 1.4 ml of DSPC in CHCl₃ solution (7 mg, 5 mg/ml), 0.7 ml of cholesterol in CHCl₃ solution (3.5 mg, 5 mg/ml), 1.4 ml of D, L-erythro-6-glucose-corynomycolate in CHCl₃ solution (1.4 mg, 1.0 mg/ml), and 1.4 ml of D, L-threo-6-glucose-corynomycolate in CHCl₃ solution (1.4 mg, 1.0 mg/ml) were dried down by N₂ stream in a sterilized test tube. Then 2.1 ml PBS was added. The mixtures were heated again for 5 min. in a 65° C. water bath and then sonicated again for 5-10 min. at 50° C. (occasionally vortexed). The sonicated solution was extruded at 65-7° C. through an 800 nm membrane for 11 times (The membranes, filters, and extruder were sterilized). To the extruded solution, 0.7 ml of QS-21 solution (0.7 mg, 1.0 mg/ml in PBS) was added (total volume 2.8 ml, 14 dose, 0.2 ml/dose). The final dose of each of the vaccine components was 1 mg of lipid antigen, 0.5 mg of DSPC, 0.25 mg of cholesterol, 0.1 mg of D, L-erythro-6-glucose-corynomycolate, 0.1 mg of D, L-threo-6-glucose-corynomycolate, and 0.05 mg of QS-21.

Vaccine formulations prepared according to the above procedure exhibit enhanced incorporation of lipid antigens as compared to vaccines prepared without carrier lipid, and extruded vaccine formulations advantageously exhibit a more uniform particle size with respect to vaccines prepared without an extrusion step.

What we claim:

1. A method of enhancing an immune response in a mammal to Mycobacterium tuberculosis comprising the step of administering to the mammal an effective amount of a vaccine composition comprising at least one isolated and purified Mycobacterium tuberculosis nonpeptide antigen.

2. The method of claim 1 wherein the vaccine composition further comprises at least one T-cell stimulating compound.

3. The method of claim 2 wherein the T-cell stimulating compound is an adjuvant.

4. The method of claim 3 wherein the adjuvant is selected from mineral salt adjuvants, Incomplete or Complete Freund's Adjuvant, Bacille Calmette-Guerin adjuvant, block polymer adjuvant, cholera toxin, cytokine, CpG motif-containing adjuvant, oil/water emulsion adjuvants, MF-59 adjuvants, LeIF adjuvants, liposome adjuvant, ISCOM adjuvant, Monohosphoryl lipid A adjuvant, biodegradable microsphere adjuvant, muramyl dipeptide adjuvant, polyphosophazene adjuvants, and saponin adjuvants.

5. The method of claim 4 wherein the saponin adjuvant is selected from QS-7, QS-17, QS-18, or QS-21.

6. The method of claim 2 wherein the T-cell stimulating compound is a protein carrier that stimulates Class II MHC.

7. The method of claim 6 wherein the protein carrier is selected from keyhole limpet hemocyanin, diptheria toxoid, and tetanus toxoid.

8. The method of claim 1 wherein the nonpeptide antigen is selected from 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, NA-2-17, a compound of formula I, a compound of formula II, a compound of formula III, a compound of formula IV, a compound of formula V, or a compound of formula VI.

9. The method of claim 1 wherein the mammal is a human.

10. A vaccine composition comprising at least one nonpeptide antigen isolated and purified from Mycobacterium tuberculosis, wherein the vaccine composition enhances an immune response to Mycobacterium tuberculosis in a mammal to which the vaccine is administered.

11. The vaccine composition of claim 10 further comprising a T-cell stimulating compound.

12. The vaccine composition of claim 11 wherein the T-cell stimulating compound is an adjuvant.

13. The vaccine composition of claim 12 wherein the adjuvant is selected from mineral salt adjuvants, Incomplete or Complete Freund's Adjuvant, Bacille Calmette-Guerin adjuvant, block polymer adjuvant, cholera toxin, cytokine, CpG motif-containing adjuvant, oil/water emulsion adjuvants, MF-59 adjuvants, LeIF adjuvants, liposome adjuvant, ISCOM adjuvant, Monohosphoryl lipid A adjuvant, biodegradable microsphere adjuvant, muramyl dipeptide adjuvant, polyphosophazene adjuvants, and saponin adjuvant.

14. The vaccine composition of claim 13 wherein the saponin adjuvant is selected from QS-7, QS-17, QS-18, or QS-21.

15. The vaccine composition of claim 11 wherein the T-cell stimulating compound is a protein carrier.

16. The vaccine composition of claim 15 wherein the protein carrier is selected from keyhole limpet hemocyanin, diptheria toxoid, and tetanus toxoid.

17. The vaccine composition of claim 10 wherein the nonpeptide antigen is selected from 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, NA-2-17, a compound of formula I, a compound of formula II, a compound of formula III, a compound of formula IV, a compound of formula V, or a compound of formula VI.

18. A pharmaceutical composition comprising:
a. at least one nonpeptide antigen isolated and purified from Mycobacterium tuberculosis, and
b. a vehicle.

19. The pharmaceutical composition of claim 18 further comprising at least one T-cell stimulating compound.

20. The pharmaceutical composition of claim 19 wherein the T-cell stimulating compound is an adjuvant.

21. The pharmaceutical composition of claim 20 wherein the adjuvant is selected from mineral salt adjuvants, Incomplete or Complete Freund's Adjuvant, Bacille Calmette-Guerin adjuvant, block polymer adjuvant, cholera toxin, cytokine, CpG motif-containing adjuvant, oil/water emulsion adjuvants, MF-59 adjuvants, LeIF adjuvants, liposome adjuvant, ISCOM adjuvant, Monohosphoryl lipid A adjuvant, biodegradable microsphere adjuvant, muramyl dipeptide adjuvant, polyphosophazene adjuvants, and saponin adjuvants.

22. The pharmaceutical composition of claim 21 wherein the saponin adjuvant is selected from QS-7, QS-17, QS-18, or QS-21.

23. The pharmaceutical composition of claim 18 wherein the nonpeptide antigen is selected 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, NA-2-17, a compound of formula I, a compound of formula II, a compound of formula III, a compound of formula IV, a compound of formula V, or a compound of formula VI.

24. The pharmaceutical composition of claim 19 wherein the T-cell stimulating compound is a protein carrier capable of stimulating Class II MHC.

25. The pharmaceutical composition of claim 24 wherein the protein carrier is selected from keyhole limpet hemocyanin, diptheria toxoid, and tetanus toxoid.

26. A nonpeptide antigen isolated and purified from Mycobacterium tuberculosis.

27. The nonpeptide antigen of claim 26 selected from a 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, NA-2-17, a compound of formula I, a compound of formula II, a compound of formula III, a compound of formula IV, a compound of formula V, or a compound of formula VI.

28. A vaccine composition comprising one or more nonpeptide antigen isolated and purified from Mycobacterium tuberculosis and at least one lipid carrier, wherein the vaccine composition comprises lipid vesicles.

29. The composition of claim 28, wherein the lipid carrier and the nonpeptide antigen are at a ratio of between 1:10 to 20:1.

30. The composition of claim 28, wherein the lipid vesicles have a radius of less than 1000 nm.

31. The composition of claim 28, wherein the average radius of the lipid vesicles is between 50 and 500 nm.

32. A method of making a vaccine composition, comprising the step of extruding a mixture comprising one or more lipid carriers and one or more isolated nonpeptide antigens through a filter membrane.

33. The method of claim 32, wherein the lipid carrier and the nonpeptide antigen are at a ratio of between 1:10 to 20:1.

34. The method of claim 32, wherein the filter membrane has a pore size of between 50 nm and 2000 nm.

35. The composition of claim 28, wherein the one or more nonpeptide antigen is 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, NA-2-17, a compound of formula I, a compound of formula II, a compound of formula III, a compound of formula IV, a compound of formula V, or a compound of formula VI.

36. The method of claim 32, wherein the one or more nonpeptide antigens is 2640-35A, 2640-38C, 2640-47E, 2640-47F, 2640-47H, 2640-48D, M-2-1, M-2-2, M-2-3, M-2-4, M-2-5, M-2-6, M-2-7, M-2-8, M-2-9, M-2-10, M-2-11, M-2-12, NA-2-1, NA-2-2, NA-2-3, NA-2-4, NA-2-5, NA-2-6, NA-2-7, NA-2-8, NA-2-9, NA-2-10, NA-2-11, NA-2-12, NA-2-13, NA-2-14, NA-2-15, NA-2-16, NA-2-17, a compound of formula I, a compound of formula II, a compound of formula III, a compound of formula IV, a compound of formula V, or a compound of formula VI.

37. The composition of claim 28, further comprising an adjuvant.

38. The method of claim 32, further comprising an adjuvant.
